(19) United States
(12) Patent Application Publication
(10) Pub. No.: US 2008/0279802 A1
(43) Pub. Date: Nov. 13, 2008

(54) PERMANENT RESHAPING OF COLORED FIBERS CONTAINING KERATIN

(76) Inventors: Burkhard MULLER, Hamburg (DE); Meike LUDWIG, Hamburg (DE); Thorsten KNAPPE, Schenefeld (DE)

Correspondence Address: PAUL & PAUL, 2000 MARKET STREET, Suite 2900, PHILADELPHIA, PA, 19103-3229, US

(21) Appl. No.: 12/139,463

(22) Filed: Jun. 14, 2008

Related U.S. Application Data
(63) Continuation of application No. PCT/EP2006/011173, filed on Nov. 22, 2006.

(30) Foreign Application Priority Data
Dec. 19, 2005 (DE) 10 2005 061 022.6

(51) Int. Cl. A61K 8/64; A61Q 5/04

(52) U.S. Cl. 424/70.2

(57) ABSTRACT

A process for improving the color stability of colored fibers containing keratin, in particular human hair, during the permanent shaping of fibers containing keratin, compositions suitable for this purpose comprising at least one silk protein hydrolyzate derivatized with at least one fatty acid and at least one keratin-reducing compound, and also a process for the permanent shaping of colored fibers containing keratin using the compositions.

### CROSS-REFERENCE TO RELATED APPLICATIONS

This application is a continuation under 35 U.S.C. Section 365(c) and 35 U.S.C. Section 120 of International Application No. PCT/EP2006/011173, filed Nov. 22, 2006. This application also claims priority under 35 U.S.C. Section 119 of German Patent Application No. DE 10 2005 061 002.6, filed Dec. 19, 2005. Both the International Application and the German Application are incorporated herein by reference in their entireties.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The invention relates to the use of specific protein hydrolyzate derivatives for improving the color stability of colored fibers containing keratin, in particular human hair, during the permanent shaping of fibers containing keratin, compositions suitable for this purpose, and to a method for permanently shaping colored fibers containing keratin using the compositions.

Fibers containing keratin that may be used are in principle all animal hair, e.g., wool, horsehair, angora hair, furs, feathers and products or textiles made from these. Preferably, however, the keratin fibers are human hair and wigs made therefrom.

Permanent shaping of fibers containing keratin is usually carried out by mechanically shaping the fibers and fixing the shape using suitable auxiliaries. Before and/or after this shaping, the fibers are treated with a keratin-reducing preparation. After a rinsing operation, the fiber is then treated in a so-called neutralizing step, with an oxidizing agent preparation, rinsed and, after or during the neutralizing step, freed from shaping auxiliaries (rollers, papillotes). If the keratin-reducing component used is a mercaptan, e.g., ammonium thioglycolate, this cleaves some of the disulfide bridges in the keratin molecule to —SH groups, resulting in a softening of the keratin fiber. During the subsequent oxidative neutralization, disulfide bridges in the hair keratin are joined again so that the keratin structure is fixed in the pregiven shape. Alternatively, it is known to use sulfite instead of the mercaptans for shaping hair. Through hydrogen sulfite solutions and/or sulfite solutions and/or disulfite solutions, disulfite bridges of keratin are cleaved in a sulfitolysis according to the equation

R—S—S—R+HSO₃⁽⁻⁾→R—SH+R—S—SO₃⁽⁻⁾

and in this way softening of the keratin fibers is achieved. Reducing agents containing hydrogen sulfite, sulfite or disulfite do not have the strong intrinsic odor of the agents containing mercaptan. The cleavage can be reversed again, as described above, in a neutralizing step with the help of an oxidizing agent to form new disulfide bridges.

The permanent smoothing of fibers containing keratin is achieved analogously through the use of keratin-reducing and -oxidizing compositions. In a corresponding method, the curly hair is either wound onto rollers with a large diameter of usually more than 15 mm, or the hair is combed smooth under the action of the keratin-reducing composition. Instead of the roller, it is also possible to smooth the fibers on a smoothing board. Smoothing boards are usually rectangular plates made, for example, of plastic.

Particular problems occur during the permanent shaping of colored fibers containing keratin. Firstly, the fibers are already stressed and optionally pre-damaged as a result of the coloring operation. During permanent shaping, it must therefore be ensured that the fibers are treated as gently as possible and that a uniform reshaping result is obtained. Secondly, the coloration is not completely stable toward conventional reshaping compositions. The result is destruction and/or the leaching of the dyes, the color fades and even the nuance changes.

In order to keep damage to the fibers containing keratin through permanent shaping as low as possible, it has already been proposed on numerous occasions to add a conditioning compound to the keratin-reducing composition and/or to the neutralizer. The use of a large number of such compounds, and of mixtures of various conditioning compounds is known.

(2) Description of Related Art, Including Information Disclosed Under 37 C.F.R. Sections 1.97 and 1.98

Thus, WO 2005/020943 A1 discloses a method of smoothing fibers containing keratin, where the keratin-reducing composition and/or the oxidizing agent composition comprises at least one conditioning compound selected from cationic polymers, quaternary ammonium compounds, silicones and protein hydrolyzates. As regards suitable protein hydrolyzates, no particular limitations are imposed. The use of derivatives of the protein hydrolyzates is also mentioned summarily. The addition of these conditioners prevents in particular damage to the fibers during the heat treatment customary in smoothing methods. The problem of color loss during permanent reshaping of colored fibers is not discussed. This problem remains unsolved.

### BRIEF SUMMARY OF THE INVENTION

It is therefore an object of the invention to provide a procedure which allows colored fibers containing keratin to be permanently shaped while largely retaining the color.

Surprisingly, it has been found that the object can be achieved through the use of protein hydrolyzates derivatized with fatty acids.

The invention therefore firstly provides the use of protein hydrolyzates derivatized with at least one fatty acid for improving the color stability of colored fibers containing keratin, in particular human hair, during the permanent shaping of fibers containing keratin.

Derivatization of the protein hydrolyzates can take place in a known manner by reacting the desired protein hydrolyzate with fatty acids or fatty acid derivatives, in particular fatty acid halides, for example, the fatty acid chlorides.

Preference is given to using protein hydrolyzates which are derivatized with at least one C₆-C₃₀-fatty acid, preferably with at least one C₁₀-C₂₀-fatty acid, particularly preferably with at least one C₁₂-C₁₈-fatty acid. Mixtures of different fatty acids can of course also be used for the derivatization.

Suitable protein hydrolyzates derivatized with fatty acids can be derived from protein hydrolyzates both of vegetable and also animal or marine or synthetic origin.

Protein hydrolyzates of vegetable origin are, for example, soy, almond, pea, potato and wheat protein hydrolyzates. Such products are available, for example, under the trade names Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Animal protein hydrolyzates are, for example, elastin, collagen, keratin, silk and milk protein hydrolyzates, which may also be present in the form of salts. Such products are sold, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm) and Kerasol® (Croda).

### BRIEF DESCRIPTION OF THE SEVERAL VIEWS OF THE DRAWING(S)

Not Applicable

### DETAILED DESCRIPTION OF THE INVENTION

Preference is given to using protein hydrolyzates derivatized with fatty acids that are derived from protein hydrolyzates of animal origin.

Of particular interest is the use of derivatized silk protein hydrolyzates since these result in a particularly marked improvement in the color stability of colored hair during permanent reshaping.

Silk is understood as meaning the fibers of the cocoon of the mulberry silk worm (Bombyx mori L.). The crude silk fiber consists of a double thread of fibroin. The cementing substance holding these double fibers together is sericin. Silk consists of 70-80% by weight of fibroin, 19-28% by weight of sericin, 0.5-1% by weight of fat and 0.5-1% by weight of dyes and mineral constituents.

The essential constituents of sericin are, at about 46% by weight, hydroxy amino acids. Sericin consists of a group of 5 to 6 proteins. The essential amino acids of sericin are serine (Ser, 37% by weight), aspartate (Asp, 26% by weight), glycine (Gly, 17% by weight), alanine (Ala), leucine (Leu) and tyrosine (Tyr).

Water-insoluble fibroin is a type of scleroprotein with a long-chain molecular structure. The main constituents of fibroin are glycine (44% by weight), alanine (26% by weight), and tyrosine (13% by weight). A further essential structural feature of fibroin is the hexapeptide sequence Ser-Gly-Ala-Gly-Ala-Gly.

Technically, it is possible, in a simple manner, to separate the two silk proteins from one another. It is therefore of no surprise that both sericin and also fibroin are each known in their own right as raw materials for use in cosmetic products. Furthermore, protein hydrolyzates and derivatives based on the individual silk proteins in each case are known raw materials in cosmetic compositions. Thus, for example, sericin is sold as such by Pentapharm Ltd. as a commercial product with the name Sericin Code 303-02. Yet much more frequently, fibroin is supplied commercially as protein hydrolyzate with various molecular weights. These hydrolyzates are sold in particular as “silk hydrolyzates”. Thus, for example, silk hydrolyzed fibroin with average molecular weights between 350 and 1000 is sold under the trade name Promois®. For the purposes of the invention, such hydrolyzates are encompassed by the term silk protein hydrolyzate.

Particular preference is given to using protein hydrolyzates derivatized with at least one fatty acid which are selected from the compounds with the INCI names cocoyl hydrolyzed silk, potassium cocoyl hydrolyzed silk, sodium cocoyl hydrolyzed silk, isostearoyl hydrolyzed silk, AMP-isostearoyl hydrolyzed silk, sodium lauroyl hydrolyzed silk, sodium stearoyl hydrolyzed silk and mixtures thereof.

Very particular preference is given to use of sodium lauroyl hydrolyzed silk, as is marketed, for example, by Seiwa Kasei under the name Promois EFLS.

According to the invention, it is also possible to use a mixture of two or more fatty acid-derivatized protein hydrolyzates.

Preference is given to using the protein hydrolyzates derivatized with a fatty acid in the form of a composition which comprises the protein hydrolyzates derivatized with a fatty acid in concentrations of from 0.05% by weight to 20% by weight, particularly preferably from 0.1% by weight to 15% by weight and very particularly preferably in amounts of from 0.5% by weight to 5% by weight, in each case based on the total composition.

Particular preference is given to using the protein hydrolyzates derivatized with a fatty acid in the form of an aqueous composition comprising the protein hydrolyzate derivatized with at least one fatty acid and at least one keratin-reducing compound.

An aqueous composition for the purposes of the invention comprises at least 50% by weight of water, based on the weight of the total composition.

The keratin-reducing compounds here are preferably selected from compounds with at least one thiol group and derivatives thereof, from sulfites, hydrogen sulfites and disulfites.

Compounds with at least one thiol group and derivatives thereof are, for example, thioglycolic acid, thiolactic acid, thiomalic acid, phenylthioglycolic acid, mercaptoethanesulfonic acid and salts and esters thereof (such as, for example, isooctyl thioglycolate and isopropyl thioglycolate), cysteamine, cystein, Bunte salts and salts of sulfurous acid. Of particular suitability are the monoethanolammonium salts or ammonium salts of thioglycolic acid and/or of thiolactic acid, and also the free acids. These are used in the aqueous composition preferably in concentrations of from 0.5 to 2.0 mol/kg at a pH of from 5 to 12, in particular from 7 to 9.5. To establish this pH, the aqueous compositions usually comprise alkalizing agents such as ammonia, alkali metal and ammonium carbonates and hydrogen carbonates or organic amines such as monoethanolamine.

Examples of keratin-reducing compounds of the disulfites which may be present in the aqueous composition are alkali metal disulfites, such as, for example, sodium disulfite (Na₂S₂O₅) and potassium disulfite (K₂S₂O₅), and also magnesium disulfite and ammonium disulfite ((NH₄)₂S₂O₅). According to the invention, ammonium disulfite may be preferred here. Examples of keratin-reducing compounds of the hydrogen sulfites which may be present in the aqueous composition are hydrogen sulfites as alkali metal, magnesium, ammonium or alkanolammonium salt based on a C₂-C₄-mono-, di- or trialkanolamine. Ammonium hydrogen sulfite may here be a particularly preferred hydrogen sulfite. Examples of keratin-reducing compounds of the sulfites which may be present in the aqueous composition are sulfites as alkali metal, ammonium or alkanolammonium salt based on a C₂-C₄-mono-, di- or trialkanolamine.

Ammonium sulfite is preferred here. The pH of the aqueous composition is adjusted when using sulfite and/or disulfite and/or hydrogen sulfite preferably to a value in the neutral range from pH 5 to 8, preferably from pH 6 to 7.5.

According to the invention, preferred C₂-C₄-alkanolamines are 2-aminoethanol (monoethanolamine) and N,N,N-tris(2-hydroxyethyl)amine (triethanolamine). Monoethanolamine is a particularly preferred C₂-C₄-alkanolamine, which is used in particular in an amount of from 0.2 to 6% by weight, based on the total aqueous composition.

The keratin-reducing compounds present in the aqueous composition are particularly preferably selected from thioglycolic acid, thiolactic acid and cystein, and salts thereof.

The keratin-reducing compound is preferably used in an amount of from 1 to 25% by weight, particularly preferably in an amount of from 5 to 15% by weight, based on the total aqueous, keratin-reducing composition.

Moreover, the aqueous, keratin-reducing composition can comprise further components which promote the effect of the keratin-reducing compound on the keratin. Such components are, for example, swelling agents for fibers containing keratin, such as, for example, C₁-C₆-alcohols and water-soluble glycols or polyols, such as, for example, glycerol, 1,2-propylene glycol or sorbitol and urea or urea derivatives, such as, for example, allantoin and guanidine, and also imidazole and derivatives thereof. In one preferred embodiment, the aqueous composition comprises 0.05 to 5% by weight of 1,2-propylene glycol and/or 0.05 to 5% by weight of urea. The quantitative data refer in each case to the total aqueous composition.

If the protein hydrolyzates derivatized with at least one fatty acid are used in the form of a composition, in particular in the form of a composition which furthermore comprises at least one keratin-reducing compound, then the composition can furthermore comprise the known active ingredients, auxiliaries and additives which are customarily added to waving or smoothing compositions.

Thus, the compositions can, for example, comprise at least one viscosity-increasing compound, referred to below as thickener.

Thickeners that can be used according to the invention are, for example, agar agar, guar gum, alginates, xanthan gum, gum Arabic, karaya gum, carob seed flour, linseed gums, dextrans, cellulose derivatives, e.g., methylcellulose, hydroxyalkylcellulose and carboxymethylcellulose, starch fractions and derivatives such as amylose, amylopectin and dextrins, clays, such as, for example, bentonite, or completely synthetic hydrocolloids, such as, for example, polyvinyl alcohol, and also viscosity-increasing polymers based on polyacrylate, as are sold, for example, under the trade names Pemulen®, Aculyn® and Carbopol®. Furthermore, preference is given to using a mixture of diesters of 1,2-propylene glycol with fatty acids, for example the thickener with the INCI name Propylene Glycol Dicaprylate/Dicaprate.

The composition can be present in one of the customary forms, for example in the form of a cream, a lotion or an emulsion, for example an oil-in-water emulsion (O/W emulsion), a water-in-oil emulsion (W/O emulsion) or a multiple emulsion.

Emulsions are generally understood as meaning heterogeneous systems which consist of two liquids that are immiscible or of only limited miscibility with one another, these usually being referred to as phases. In an emulsion, one of the liquids is dispersed in the form of fine droplets in the other liquid with expenditure of energy to create stabilizing phase interfaces. Emulsions are known in which permanent dispersion of one liquid in another liquid can be achieved without the addition of further auxiliaries. However, it is generally advisable to stabilize emulsions by adding so-called emulsifiers.

The composition in which the protein hydrolyzates derivatized with at least one fatty acid are used can therefore furthermore comprise at least one emulsifier. Emulsifiers bring about, at the phase interface, the formation of water- or oil-stable adsorption layers which protect the dispersed droplets against coalescence and thus stabilize the emulsion. Emulsifiers are therefore composed like surfactants from a hydrophobic molecular moiety and a hydrophilic molecular moiety. Hydrophilic emulsifiers form preferably O/W emulsions and hydrophobic emulsifiers form preferably W/O emulsions. Selection of these emulsifying surfactants or emulsifiers is governed here by the substances to be dispersed and the particular external phase and also the finely divided nature of the emulsion. More detailed definitions and properties of emulsifiers can be found in “H.-D. Dörfler, Grenzflächen-und Kolloidchemie, [Interface and colloid chemistry], VCH Verlagsgesellschaft mbH. Weinheim 1994”. Emulsifiers that can be used according to the invention are, for example,
addition products of from 4 to 100 mol of ethylene oxide and/or 1 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,
C₁₂-C₂₂-fatty acid mono- and diesters of addition products of from 1 to 30 mol of ethylene oxide onto polyols having 3 to 6 carbon atoms, in particular onto glycerol,
ethylene oxide and polyglycerol addition products onto methyl glucoside fatty acid esters, fatty acid alkanolamides and fatty acid glucamides,
C₈-C₂₂-alkyl mono- and oligoglycosides and ethyoxylated analogs thereof, where degrees of oligomerization of from 1.1 to 5, in particular 1.2 to 2.0, and glucose are preferred as sugar component,
mixtures of alkyl (oligo)glucosides and fatty alcohols, for example the commercially available product Montanov® 68,
addition products of from 5 to 60 mol of ethylene oxide onto castor oil and hydrogenated castor oil,
partial esters of polyols having 3-6 carbon atoms with saturated fatty acids having 8 to 22 carbon atoms,
sterols. Sterols are understood as meaning a group of steroids which carry a hydroxyl group on carbon atom 3 of the steroid backbone and are isolated either from animal tissue (zoosterols) or from vegetable fats (phytosterols). Examples of zoosterols are cholesterol and lanosterol. Examples of suitable phytosterols are ergosterol, stigmasterol and sitosterol. Sterols are also isolated from fungi and yeasts, these being the so-called mycosterols.
Phospholipids. These are understood primarily as meaning the glucose phospholipids which are obtained, for example, as lecithins or phosphahtidylcholines from, for example, egg yolk or plant seeds (e.g., soybeans).
Fatty acid esters of sugars and sugar alcohols, such as sorbitol,
polyglycerols and polyglycerol derivatives, such as, for example, polyglycerol poly-12-hydroxystearate (commercial product Dehymuls® PGPH),
linear and branched fatty acids having 8 to 30 carbon atoms and the Na—, K, ammonium, Ca, Mg and Zn salts thereof.

The emulsifiers are preferably used in amounts of from 0.1 to 25% by weight, in particular 0.1 to 3% by weight, based on the respective total composition.

Preference is given to nonionogenic emulsifiers with an HLB value of 8 to 18, according to the definitions listed in the Römpp Lexikon of Chemistry (ed. J. Falbe, M. Regitz), 10th edition, Georg Thieme Verlag Stuttgart, New York (1997), page 1764. Nonionogenic emulsifiers with an HLB value of from 10 to 16 are particularly preferred according to the invention.

Furthermore, the compositions can comprise at least one oil, with both natural and synthetic oils, such as, for example, vegetable oils, liquid paraffin oils, but also ester oils, dicarboxylic acid esters, symmetrical, asymmetrical or cyclic esters of carbonic acid with fatty alcohols, trifatty acid esters of fatty acids with glycerol, fatty acid partial glycerides and fatty alcohols being suitable.

The oil is preferably a linear or branched, saturated or unsaturated fatty alcohol. Fatty alcohols that can be used are fatty alcohols with C₆-C₃₀, preferably C₁₀-C₂₂ and very particularly preferably C₁₂-C₂₂ groups. For the purposes of the invention, it is possible to use, for example, decanol, octanol, octenol, dodecenol, decenol, octadienol, dodecadienol, decadienol, oleyl alcohol, eruca alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, and also the Guerbet alcohols thereof, the intention being for this list to be exemplary and nonlimiting in character. However, the fatty alcohols originate from preferably natural fatty acids, in which case it may be customary to start from an isolation from the esters of the fatty acids by reduction. According to the invention it is likewise possible to use those fatty alcohol cuts which are produced by reduction of naturally occurring triglycerides, such as beef tallow, palm oil, peanut oil, rapeseed oil, cottonseed oil, soy oil, sunflower oil or linseed oil or fatty acid esters that form from their transesterification products with corresponding alcohols, and thus constitute a mixture of different fatty alcohols. Such substances are commercially available, for example, under the names Stenol®, e.g., Stenol® 1618 or Lanette®, e.g., Lanette® 0 or Lorol®, e.g., Lorol® C8, Lorol® C14, Lorol® C18, Lorol® C8-18, HD-Ocenol®, Crodacol®, e.g., Crodacol® CS, Novol®, Eutanol® G, Guerbitol® 16, Guerbitol® 18, Guerbitol®20, Isofol® 12, Isofol® 16, Isofol®24, Isofol®36, Isocarb® 12, Isocarb® 16 or Isocarb® 24. According to the invention it is also of course possible to use wool wax alcohols, as are commercially available, for example, under the name Corona®, White Swan®, Coronet® or Fluilan®. Particular preference is given to using mixtures of stearyl alcohol and cetyl alcohol, which are referred to in INCI nomenclature as Cetearyl Alcohol.

The fatty alcohols are used, for example, in amounts of from 0.1 to 20% by weight, based on the total preparation, preferably in amounts of from 0.1 to 10% by weight.

The composition can furthermore comprise, for example, conditioners, surfactants, UV stabilizers, complexing agents or preservatives.

Suitable surfactants are all surface-active substances from the group of nonionic, anonic, and amphoteric surfactants, where the group of amophoteric or else ampholytic surfactants includes zwitterionic surfactants and ampholytics. These surfactants have, inter alia, the task of promoting the wetting of the keratin surface by the treatment solution. The surfactants can also have an emulsifying effect.

Suitable anionic surfactants are in principle all anionic surface-active substances that are suitable for use on the human body. These are characterized by a water-solubilizing, anionic group, such as, for example, a carboxylate, sulfate, sulfonate or phosphate group and a lipophilic alkyl group having about 8 to 30 carbon atoms. Additionally, glycol or polyglycol ether groups, ester, ether and amide groups, and also hydroxyl groups may be present in the molecule. Examples of suitable anionic surfactants are, in each case in the form of the sodium, potassium and ammonium and also the mono-, di- and trialkanolammonium salts having 2 to 4 carbon atoms in the alkanol group:
linear and branched fatty acids having 8 to 30 carbon atoms (soaps)
ether carboxylic acids of the formula R—O—(CH₂—CH₂—O)ₓ—CH₂—COOH, in which R is a linear alkyl group having 8 to 30 carbon atoms and x=0 or 1 to 16;
acyl sarcosides having 8 to 24 carbon atoms in the acyl group;
acyl taurides having 8 to 24 carbon atoms in the acyl group;
acyl isethionates having 8 to 24 carbon atoms in the acyl group;
sulfosuccinic acid mono- and dialkyl esters having 8 to 24 carbon atoms in the alkyl group and sulfosuccinic acid monoalkyl polyoxyethyl esters having 8 to 24 carbon atoms in the alkyl group and 1 to 6 oxyethyl groups;
linear alkanesulfonates having 8 to 24 carbon atoms;
linear alpha-olefinsulfonates having 8 to 24 carbon atoms;
alpha-sulfo fatty acid methyl esters of fatty acids having 8 to 30 carbon atoms;
alkyl sulfates and alkyl polyglycol ether sulfates of the formula R—O(CH₂₂O)ₓ—OSO₃H, in which R is a preferably linear alkyl group having 8 to 30 carbon atoms and x=0 or 1 to 12;
mixtures of surface-active hydroxysulfonates as in DE-A-37 25 030;
sulfated hydroxyalkyl polyethylene and/or hydroxyalkylene propylene glycol ethers as in DE-A-37 23 354;
sulfonates of unsaturated fatty acids having 8 to 24 carbon atoms and 1 to 6 double bonds as in DE-A-39 26 344;
esters of tartaric acid and citric acid with alcohols which constitute addition products of about 2-15 molecules of ethylene oxide and/or propylene oxide onto fatty alcohols having 8 to 22 carbon atoms;
alkyl and/or alkenyl ether phosphates of the formula (E1-I)

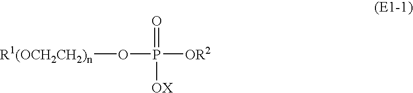

in which R¹ is preferably an aliphatic hydrocarbon radical having 8 to 30 carbon atoms, R² is hydrogen, a radical (CH₂CH₂O)ₙR¹ or X, n is numbers from 1 to 10 and X is hydrogen, an alkali metal or alkaline earth metal or NR³R⁴R⁵R⁶, where R³ to R⁶, independently of one another, are hydrogen or a C1 to C4-hydrocarbon radical;
sulfated fatty acid alkylene glycol esters of the formula (E1-II)

R⁷CO(AlkO)ₙSO₃M (E1-II);
in which R⁷CO— is a linear or branched, aliphatic, saturated and/or unsaturated acyl radical having 6 to 22 carbon atoms, Alk is CH₂CH₂, CHCH₃CH₂ and/or CH₂CHCH₃, n is numbers from 0.5 to 5 and M is a cation, as are described in DE-A 197 36 906,
monoglyceride sulfates and monoglyceride ether sulfates of the formula (E1-III)

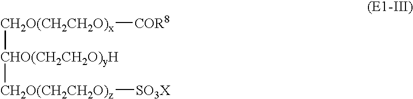

in which R⁸CO is a linear or branched acyl radical having 6 to 22 carbon atoms, x, y and z are in total 0 or numbers from 1 to 30, preferably 2 to 10, and X is an alkali metal or alkaline earth metal. Typical examples of monoglyceride (ether) sulfates suitable for the purposes of the invention are the reaction products of lauric acid monoglyceride, coconut fatty acid monoglyceride, palmitic acid monoglyceride, stearic acid monoglyceride, oleic acid monoglyceride and tallow fatty acid monoglyceride, and also ethylene oxide adducts thereof with sulfur trioxide or chlorosulfonic acid in the form of their sodium salts. Preference is given to using monoglyceride sulfates of the formula (E1-III) in which R³CO is a linear acyl radical having 8 to 18 carbon atoms, as have been described, for example, in EP 0 561 825 B1, EP 0 561 999 B1, DE 42 04 700 A1 or by A. K. Biswas et al. in J. Am. Oil. Chem. Soc. 37, 171 (1960) and F. U. Ahmed in J. Am. Oil. Chem. Soc. 67, 8 (1990);
amide ether carboxylic acids as described in EP 0 690 044;
condensation products of C₈-C₃₀-fatty alcohols with protein hydrolyzates and/or amino acids and derivatives thereof which are known to the person skilled in the art as protein fatty acid condensates, such as, for example, the Lamepon® grades, Gluadin® grades, Hostapon® KCG or the Amisoft® grades.

Preferred anionic surfactants are alkyl sulfates, alkyl polyglycol ether sulfates and ether carboxylic acids having 10 to 18 carbon atoms in the alkyl group and up to 12 glycol ether groups in the molecule, sulfosuccinic acid mono- and dialkyl esters having 8 to 18 carbon atoms in the alkyl group and sulfosuccinic acid monoalkyl polyoxyethyl esters having 8 to 18 carbon atoms in the alkyl group and 1 to 6 oxyethyl groups, monoglyceride sulfates, alkyl and alkenyl ether phosphates and protein fatty acid condensates.

Zwitterionic surfactants is the term used to refer to those surface-active compounds which carry at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example. cocoacylaminopropyldimethylammonium glycinate, and 2-alkyl-3-carboxymethyl-3-hydroxyethylimidazolines having in each case 8 to 18 carbon atoms in the alkyl or acyl group, and also cocoacylaminoethyl hydroxyethylcarboxymethylglycinate. A preferred zwitterionic surfactant is the fatty acid amide derivative known under the INCI name Cocamidopropyl Betaine.

Ampholytics are understood as meaning those surface-active compounds which, apart from a C₈-C₂₄-alkyl or acyl group in the molecule, contain at least one free amino group and at least one —COOH or —SO₃H group and are capable of forming internal salts. Examples of suitable ampholytics are N-alkylglycines, N-alkylpropionic acids, N-alkylaminobutteric acids, N-alkyliminodipropionic acids, N-hydroxyethyl-N-alkylamidopropylglycines, N-alkyltraurines, N-alkylsarcosines, 2-alkylaminopropionic acids and alkylaminoacetic acids having in each case about 8 to 24 carbon atoms in the alkyl group. Particularly preferred ampholytics are N-cocoalkylaminopropionate, cocoacylaminoethylaminopropionate and C₁₂-C₁₈-acylsarcosine.

Nonionic surfactants comprise, as hydrophilic group, e.g., a polyol group, a polyalkylene glycol ether group or a combination of polyol and polyglycol ether group. Such compounds are, for example:
addition products of from 2 to 50 mol of ethylene oxide and/or 1 to 5 mol of propylene oxide onto linear and branched fatty alcohols having 8 to 30 carbon atoms, onto fatty acids having 8 to 30 carbon atoms and onto alkylphenols having 8 to 15 carbon atoms in the alkyl group;
addition products, terminally capped with a methyl or C₂-C₆-alkyl radical, of from 2 to 50 mol of ethylene oxide and/or 1 to 5 mol of propylene oxide onto linear and branched fatty alcohols having 8 to 30 carbon atoms, onto fatty acids having 8 to 30 carbon atoms and onto alkylphenols having 8 to 15 carbon atoms in the alkyl group, such as, for example, the grades available under the trade names Dehydrol® LS, Dehydrol® LT (Cognis);
C₁₂-C₃₀-fatty acid mono- and diesters of addition products of from 1 to 30 mol of ethylene oxide onto glycerol;
addition products of from 5 to 60 mol of ethylene oxide onto castor oil and hydrogenated castor oil;
polyol fatty acid esters, such as, for example, the commercial product Hydagen® HSP (Cognis) or Sovermol grades (Cognis);
alkoxylated triglycerides;
alkoxylated fatty acid alkyl esters of the formula (E4-I)

R¹CO—(OCH₂CHR²)_{w}OR³ (E4-I)
in which R¹CO is a linear or branched, saturated and/or unsaturated acyl radical having 6 to 22 carbon atoms, R² is hydrogen or methyl, R³ is linear or branched alkyl radicals having 1 to 4 carbon atoms and w is numbers from 1 to 20;
amine oxides;
hydroxy mixed ethers, as are described, for example, in DE-A 19738866;
sorbitan fatty acid esters and addition products of ethylene oxide onto sorbitan fatty acid esters, such as, for example, the polysorbates;
sugar fatty acid esters and addition products of ethylene oxide onto sugar fatty acid Esters;
addition products of ethylene oxide onto fatty acid alkanolamides and fatty amines;
sugar surfactants of the alkyl and alkenyl oligoglycoside type according to formula (E4-II)

R⁴O-[G]ₚ (E4-II)
in which R⁴ is an alkyl or alkenyl radical having 4 to 22 carbon atoms, G is a sugar radical having 5 or 6 carbon atoms and p is numbers from 1 to 10. They can be obtained by the relevant methods of preparative organic chemistry. By way of representation of the extensive literature, reference may be made here to the overview paper by Biermann et al. in Starch 45, 281 (1993), B. Salka in Cosm. Toil. 108, 89 (1993), and J. Kahre et al. in SÖFW-Journal issue 8, 598 (1995).

The alkyl and alkenyl oligoglycosides can be derived from aldoses or ketoses having 5 or 6 carbon atoms, preferably from glucose. The preferred alkyl and/or alkenyl oligoglycosides are thus alkyl and/or alkenyl oligoglucosides. The index number p in the general formula (E4-II) gives the degree of oligomerization (DP), i.e. the distribution of monoglycosides and oligoglycosides, and is a number between 1 and 10. Whereas p in an individual molecule must always be an integer and here primarily can assume the values p=1 to 6, the value p for a certain alkyl oligoglycoside is an analytically determined calculated parameter which in most cases is a fraction. Preference is given to using alkyl and/or alkenyl oligoglycosides with an average degree of oligomerization p of from 1.1 to 3.0. From an applications point of view, preference is given to those alkyl and/or alkenyl oligoglycosides whose degree of oligomerization is less than 1.7 and in particular is between 1.2 and 1.4. The alkyl or alkenyl radical R⁴ can be derived from primary alcohols having 4 to 11, preferably 8 to 10, carbon atoms. Typical examples are butanol, caproic alcohol, caprylic alcohol, capric alcohol and undecyl alcohol, and technical-grade mixtures thereof, as are obtained, for example, during the hydrogenation of technical-grade fatty acid methyl esters or in the course of the hydrogenation from aldehydes from the Roelen oxo synthesis. Preference is given to alkyl oligoglucosides of chain lengths C₈-C₁₀ (DP=1 to 3) which are produced as forerunning in the distillative separation of technical-grade C₈-C₁₈-coconut fatty alcohol and can be contaminated with a fraction of less than 6% by weight of C₁₋₂-alcohol, and also alkyl oligoglucosides based on technical-grade C_{9/11}-oxo alcohols (DP=1 to 3). The alkyl or alkenyl radical R¹⁵ can furthermore also be derived from primary alcohols having 12 to 22, preferably 12 to 14, carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and technical-grade mixtures thereof which can be obtained as described above. Preference is given to alkyl oligoglucosides based on hydrogenated C_{12/14}-coconut alcohol with a DP of from 1 to 3.
sugar surfactants of the fatty acid N-alkylpolyhydroxyalkylamide type, the nonionic surfactant of the formula (E4-III),

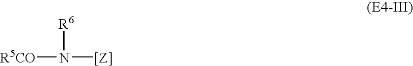

in which R⁵CO is an aliphatic acyl radical having 6 to 22 carbon atoms, R⁶ is hydrogen, an alkyl or hydroxyalkyl radical having 1 to 4 carbon atoms and [Z] is a linear or branched polyhydroxyalkyl radical having 3 to 12 carbon atoms and 3 to 10 hydroxyl groups. The fatty acid N-alkylpolyhydroxyalkylamides are known substances which can usually be obtained by reductive amination of a reducing sugar with ammonia, an alkylamine or an alkanolamine and subsequent acylation with a fatty acid, a fatty acid alkyl ester or a fatty acid chloride. With regard to the methods for their preparation, reference may be made to the US patent specifications U.S. Pat. No. 1,985,424, U.S. Pat. No. 2,016,962 and U.S. Pat. No. 2,703,798, and also the International patent application WO 92/06984. An overview of this topic by H. Kelkenberg can be found in Tens. Surf. Det. 25, 8 (1988). Preferably, the fatty acid N-alkylpolyhydroxyalkylamides are derived from reducing sugars with 5 or 6 carbon atoms, in particular from glucose. The preferred fatty acid N-alkylpolyhydroxyalkylamides are therefore fatty acid N-alkylglucamides, as are given by the formula (E4-IV):

R⁷CO—NR⁸—CH₂—(CHOH)₄CH₂OH (E4-IV)

Preferably, the fatty acid N-alkylpolyhydroxyalkylamides used are glucamides of the formula (E4-IV) in which R⁸ is hydrogen or an alkyl group and R⁷CO is the acyl radical of caproic acid, caprylic acid, capric acid, lauric acid, myristic acid, palmitic acid, palmoleic acid, stearic acid, isostearic acid, oleic acid, elaidic acid, petroselic acid, linoleic acid, linolenic acid, arachic acid, gadoleic acid, behenic acid or erucic acid or technical-grade mixtures thereof. Particular preference is given to fatty acid N-alkylglucamides of the formula (E4-IV) which are obtained by reductive amination of glucose with methylamine and subsequent acylation with lauric acid or C12/14-coconut fatty acid or a corresponding derivative. Furthermore, the polyhydroxyalkylamides can also be derived from maltose and palatinose.

Preferred nonionic surfactants have proven to be the alkylene oxide addition products onto saturated linear fatty alcohols and fatty acids having in each case 2 to 30 mol of ethylene oxide per mole of fatty alcohol or fatty acid. Preparations with excellent properties are likewise obtained if they contain fatty acid esters of ethoxylated glycerol as nonionic surfactants.

These compounds are characterized by the following parameters. The alkyl radial R contains 6 to 22 carbon atoms and may either be linear or branched. Preference is given to primary linear and 2-methyl-branched aliphatic radicals. Such alkyl radicals are, for example, 1-octyl, 1-decyl, 1-lauryl, 1-myristyl, 1-cetyl and 1-stearyl. Particular preference is given to 1-octyl, 1-decyl, 1-lauryl, 1-myristyl. When using so-called “oxo alcohols” as starting materials, compounds with an uneven number of carbon atoms in the alkyl chain predominant.

Furthermore, nonionic surfactants that may be present are the sugar surfactants. These are preferably present in amounts of from 0.1 to 20% by weight, based on the particular overall composition. Amounts of from 0.5 to 15% by weight are particularly preferred, and very particular preference is given to amounts of from 0.5 to 7.5% by weight.

The compounds with alkyl groups used as surfactant may in each case be single substances. However, it is generally preferred, when producing these substances, to start from native vegetable or animal raw materials, thus giving mixtures of substances with different alkyl chain lengths depending on the particular raw material.

In the case of the surfactants which constitute addition products of ethylene oxide and/or propylene oxide onto fatty alcohols or derivatives of these addition products, it is possible to use either products with a “normal” homolog distribution or else those with a narrowed homolog distribution. Here, “normal” homolog distribution is understood as meaning mixtures of homologs which are obtained in the reaction of fatty alcohol and alkylene oxide using alkali metals, alkali metal hydroxides or alkali metal alkoxides as catalysts. Narrowed homolog distributions on the other hand are obtained if, for example, hydro talcites, alkaline earth metal salts of ether carboxylic acids, alkaline earth metal oxides, hydroxides or alkoxides are used as catalysts. The use of products with a narrowed homolog distribution may be preferred.

The surfactants are used in amounts of from 0.1 to 45% by weight, preferably 0.5 to 30% by weight and very particularly preferably from 0.5 to 25% by weight, based on the particular overall composition used according to the invention.

Furthermore, the compositions may comprise all customary further auxiliaries and additives. For example, the following compounds may be present:
linear and/or branched fatty acids, preferably C₂-C₃₀-fatty acids, particularly preferably C₄-C₁₈ fatty acids, most preferably C₆-C₁₀-fatty acids and/or physiologically compatible salts thereof; further examples are formic acid, acetic acid, propionic acid, butyric acid, isobutyric acid, valeric acid, isovaleric acid, pivalic acid, oxalic acid, malonic acid, succinic acid, glutaric acid, lactic acid, glyceric acid, glyoxylic acid, adipic acid, pimelic acid, sorbic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluoyl acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6,7-napthalenepentaacetic acid, malonaldehydic acid, 4-hydroxyphthalamidic acid, 1-pyrazolecarboxylic acid, gallic acid or propanetricarboxylic acid,
polyhydroxy compounds; in this connection mention is to be made in particular of
sugars with 5 and/or 6 carbon atoms, in particular as mono- and/or oligosaccharides, for example glucose, fructose, galactose, lactose, arabinose, ribose, xylose, lyxose, allose, altrose, mannose, gulose, idose, tallowse and sucrose and/or derivatives thereof, e.g., ether derivatives, amino derivatives and/or acetyl derivatives, such as acetylated glucose, e.g., tetraacetylglucose, pentaecetylglucose and/or 2-acetamido-2-desoxyglucose. Preferred sugar building blocks are glucose, fructose, galactose, allose, lactose, arabinose and sucrose; glucose, galactose and lactose are particularly preferred;
aldonic acids, in particular gluconic acid, glucuronic acid;
polyols, such as, for example, glucamines, glycerol, mono- or diglycerides, 2-ethyl-1,3-hexanediol, 2-hydroxymethylpropanetriol, glycols, such as ethylene glycol, diethylene glycol, triethylene glycol, propylene glycol, dipropylene glycol, 1,3-butanediol;
polyhydroxy acids, such as, for example, pentahydroxyhexanoic acid, tetrahydroxypentanoic acid and/or derivatives thereof, such as, for example, ethers, esters and/or amides, e.g., pentahydroxyhexanoic acid amide and/or physiologically compatible salts thereof; further examples: citric acid, maleic acid or tartaric acid;
pantolactone;
panthenol and/or derivatives thereof;
further vitamins, such as, for example, vitamin B6, C and/or E and/or derivatives thereof;
hydroxy acids, such as, for example, α,β-hydroxy fatty acids and keto fatty acids and/or physiologically compatible salts thereof; such as, for example, salicylic acid or lactic acid, glyoxylic acid, glycolic acid,
water-soluble polymers setting effect, e.g., polyvinylpyrrolidone, vinyl acetate/crotonic acid copolymers,
antidandruff active ingredients, such as, for example, picrotone olamine, zinc omadine,
active ingredients, such as allantoin, pyrrolidonecarboxylic acids, plant extracts,
pH regulators and buffers, such as, for example, citric acid/sodium citrate, ammonium carbonate, ammonium hydrogencarbonate, guanidine carbonate, ammonia, sodium hydroxide,
complexing agents, such as EDTA, NTA, organophosphonic acids, dipicolinic acid, salicylic acid,
photoprotective agents (UV absorbers),
oil, fat and wax components, preferably in emulsified form,
dyes, opacifiers and pearlizing agents, and
optionally aerosol propellant gases.

Although the protein hydrolyzates derivatized with at least one fatty acid are preferably used in the form of an aqueous composition which additionally comprises at least one keratin-reducing compound, i.e., in the form of a preparation suitable for carrying out the reduction step of a customary permanent wave or hair smoothing treatment, other modes are also possible. Thus, the protein hydrolyzates derivatized with at least one fatty acid can be applied to the fibers to be treated, for example, in the course of a pre-treatment step before carrying out the reduction of the keratin fibers, in the course of an interim treatment or else in the course of a neutralizing step. Use in two or more of said steps is also possible.

The improvement in the color stability is particularly marked if the protein hydrolyzates derivatized with at least one fatty acid are used in the form of an aqueous composition which additionally comprises at least one keratin-reducing compound. As regards the protein hydrolyzates derivatized with at least one fatty acid, the use of corresponding silk protein hydrolyzates has proven useful.

Therefore, the invention secondly provides a composition for the permanent shaping of colored fibers containing keratin, comprising:
at least one silk protein hydroxylate derivatized with at least one fatty acid; and
at least one keratin-reducing compound.

With regard to the preferred silk protein hydrolyzates derivatized with at least one fatty acid, the preferred keratin-reducing compounds, the amounts and possible further ingredients, that stated above is applicable.

The invention further provides the use of a composition according to the invention in a method for permanently shaping colored fibers containing keratin.

Furthermore, the invention provides a method of permanently shaping colored fibers containing keratin, in particular human hair, where the fiber, before and/or after mechanical shaping with the help of shaping auxiliaries, is treated with an aqueous, keratin-reducing composition according to the invention, optionally after a contact time T1 is rinsed with water and/or an aqueous composition, and finally neutralized with an oxidizing composition comprising at least one oxidizing compound, and optionally after a contact time T2 is rinsed and optionally after treated.

Finally, the invention also provides a method of permanently shaping colored fibers containing keratin, in particular human hair, where:
(i) an aqueous, keratin-reducing composition according to the invention is applied to the fibers;
(ii) after a contact time T1, the fibers are rinsed and optionally dried;
(iii) the fibers are shaped with the help of shaping auxiliaries; and
(iv) finally an oxidizing composition comprising at least one oxidizing compound, is applied to the fibers and is rinsed off again after a contact time T2.

For the purposes of the methods according to the invention, shaping auxiliaries may, for example, be rollers or papillotes in the case of a permanent wave, or auxiliaries for mechanical smoothing, such as a comb or a brush, a smoothing board or heatable smoothing iron in the case of hair smoothing.

If the shaping auxiliaries, for example, rollers, are attached to the fibers in the course of a permanent waving process for a prolonged period, then it may be expedient to remove these shaping auxiliaries before, during or after applying the oxidizing composition. In this connection, it may be advantageous to leave the shaping auxiliaries in the hair while the oxidative composition acts, to remove them afterwards and then to repeat the oxidation step as a post-neutralization step.

The contact time T1 is preferably 5-60 minutes, particularly preferably 10-30 minutes. The contact time T2 is preferably 1-30 minutes, particularly preferably 1-15 minutes.

For the purposes of the invention, a dry fiber containing keratin is present if the water residues adhering to the hairs have evaporated to the extent that the hairs fall individually. Preferably, in the case of a dry fiber containing keratin, either the moisture content of the fiber is essentially in equilibrium with the moisture in the air, or the fiber absorbs moisture from the surrounding air.

By applying an oxidizing composition, the shaped fibers containing keratin are neutralized. The oxidizing compound present in the oxidizing composition has a redox potential such that two mercapto groups can be oxidized to form a disulfide bridge. A preferred oxidizing agent is selected from, for example, sodium bromate, potassium bromate or hydrogen peroxide. It is particularly preferred to use hydrogen peroxide as oxidizing agent. For stabilizing aqueous hydrogen peroxide preparations, customary stabilizers can additionally be added. The pH of the aqueous H₂O₂ preparations which, in the ready-to-use form usually comprise about 0.5 to 3.0% by weight of H₂O₂, is preferably 2 to 6. It is also possible to use concentrates with customarily up to 30% by weight of H₂O₂, which are diluted prior to use. In this connection, it may also be preferred to use standard commercial rapid neutralizers, for example Natural Styling Rinse Neutraliser 1:4 from Henkel. If the composition according to the invention comprises bromate as oxidizing agent, then this is usually present in concentrations of from 1 to 10% by weight and the pH of the solutions is adjusted to 4 to 7.

In a preferred embodiment of the methods according to the invention, the fibers containing keratin are dampened before carrying out the method. This can take place by spraying the fibers with a liquid, preferably with water. Preferably, the fibers are shampooed with a standard commercial shampoo, rinsed and then towel-dried using a hand towel. After the toweling step is complete, residual moisture can be felt in the hair. It is also possible to dampen the fibers containing keratin with a liquid which comprises at least one silk protein hydrolyzate derivatized with at least one fatty acid.

In a further preferred embodiment of the invention, the keratin fibers are subjected to a thermal treatment. It has proven particularly advantageous to carry out the thermal treatment while the aqueous, keratin-reducing composition is acting, or after rinsing out the aqueous, keratin-reducing composition.

The thermal treatment can take place, for example, by means of heatable rollers, the introduction of heated air, for example, using a hair drier or drying hood or, if the keratin fibers are to be smoothed, also with the help of appropriately heated plates, in particular metal or ceramic plates.

During the thermal treatment, the keratin fibers are preferably heated to a temperature of from 30° C. to 220° C. The preferred temperature range depends, in particular, on whether the shaping is a waving or a smoothing, and whether the thermal treatment is carried out while the aqueous, keratin-reducing composition is acting or after rinsing out the aqueous, keratin-reducing composition.

If the shaping is a waving and the thermal treatment is carried out while the aqueous, keratin-reducing composition is acting, a temperature range from 30° to 80° C., in particular from 35° C. to 60° C., is preferred.

If the shaping is a waving and if the thermal treatment is carried out after rinsing out the aqueous, keratin-reducing composition, a temperature range from 80° C. to 150° C., in particular from 80° C. to 140° C., is preferred.

If the shaping is a smoothing and if the thermal treatment is carried out while the aqueous, keratin-reducing composition is acting, temperatures from 30° C. to 80° C., in particular 35° C. to 60° C., are preferred.

If the shaping is a smoothing and if the thermal treatment is carried out after rinsing out the aqueous, keratin-reducing composition, temperatures from 120 to 220° C., in particular 130° C. to 200° C., are preferred.

After carrying out the methods according to the invention, the keratin fibers can be after-treated in the usual way. For example, the application of a standard commercial conditioner may be advantageous. Treatment with a conditioner can also take place as interim treatment.

The invention is illustrated by reference to the Examples below, the Examples being intended to facilitate the understanding of the principle according to the invention and not to be understood to be a limitation.

### EXAMPLES

A reducing agent R1 according to the invention, a comparison reducing agent C1 according to Table 1, and a neutralizer according to Table 2 were prepared.

TABLE 1
Reducing Agents.

| | Reducing Agent No. | |
|---|---|---|
| | R1 | C1 |
| Raw material | [% by weight] | [% by weight] |
| Ammonium thioglycolate (71% | 11.00 | 11.00 |
| strength aqueous solution) | | |
| Ammonia (25% strength aqueous | 1.60 | 1.60 |
| solution) | | |
| Ammonium hydrogencarbonate | 3.00 | 3.00 |
| Cremophor ® CO 40 ¹ | 1.00 | 1.00 |
| Protelan VE/K ² | 1.00 | 1.00 |
| Turpinal ® SL ³ | 0.30 | 0.30 |
| Gluadin ® WQ ⁴ | 0.20 | 0.20 |
| Polyquaternium-6 ⁵ | 0.30 | 0.30 |
| Promois EFLS ⁶ | 1.00 | — |
| Perfume | 0.50 | 0.50 |
| Water | ad 100 | ad 100 |

¹ Hydrogenated castor oil with about 40-45 EO units (INCI name: PEG-40 hydrogenated castor oil) (BASF)
² N-cocoyl wheat protein condensate (INCI name: Sodium Cocoyl Hydrolyzed Wheat Protein) (Zschimmer & Schwarz)
³ 1-hydroxyethane-1,1-diphosphonic acid (INCI name: Etidronic Acid, Aqua (Water)) (Solutia)
⁴ Wheat protein hydrolyzate (about 31-35% solids; INCI name: Aqua (Water), Laurdimonium Hydroxypropyl Hydrolyzed Wheat Protein, Ethylparaben, Methylparaben) (Cognis)
⁵ Poly(dimethyldiallylammonium chloride)
⁶ Sodium salt of the condensation product of lauroyl chloride and silk protein hydrolyzate (about 20% strength aqueous solution; INCI name: Sodium Lauroyl Hydrolyzed Silk) (Seiwa Kasei)

TABLE 2
Neutralizer.

| | F1 |
|---|---|
| Raw material | [% by weight) |
| Hydrogen peroxide | 4.00 |
| (50% strength aqueous solution) | |
| Dimethylcocoalkylamine oxide | 3.00 |
| (30% strength aqueous solution) | |
| Orthophosphoric acid | 1.00 |
| (85% strength aqueous solution) | |
| Methylparaben | 0.10 |
| Dehyquart ® A ⁷ | 0.20 |
| Polyquaternium-6 ⁵ | 0.10 |
| Perfume | 0.50 |
| Water | ad 100 |

⁷ Trimethylhexadecylammonium chloride (about 24-26% active substance; INCI name: Aqua (Water), Cetrimonium Chloride) (Cognis)

Experimental Procedure and Assessment of the Results.

### Reference.

Five hair tresses were treated with standard commercial oxidative hair color and then treated two×15 minutes in an ultrasound bath. The resulting tresses serve as reference.

### Comparison.

Five further hair tresses were colored analogously to the reference tresses, but between the first and second ultrasound treatment of 15 minutes in each case, subjected to a permanent wave treatment using the comparison reducing agent C1 as in Table 1 and the neutralizer as in Table 2. The color intensity after treatment of the tresses is considerably weaker compared to the reference tresses.

### Procedure According to the Invention.

Five further hair tresses were colored analogously to the reference tresses and between the first and second ultrasound treatment of 15 minutes in each case, subjected to a permanent wave treatment using the inventive reducing agent R1 as in Table 1 and the neutralizer as in Table 2. The reducing agent comprised the derivatized silk protein hydrolyzate Promois EFLS. The color intensity is significantly higher than the color intensity of the comparison tresses.

1. A process for improving the color stability of colored fibers containing keratin during the permanent shaping of the fibers, comprising the step of applying to the fibers protein hydrolyzates derivatized with at least one fatty acid.

2. The process as claimed in claim 1, characterized in that the protein hydrolyzates are derivatized with at least one C₆-C₃₀-fatty acid.

3. The process as claimed in claim 1, characterized in that the derivatized protein hydrolyzate is based on a protein of animal origin.

4. The process as claimed in claim 1, characterized in that the derivatized protein hydrolyzate is a silk protein hydrolyzate.

5. The process as claimed in claim 1, characterized in that the protein hydrolyzate derivatized with at least one fatty acid is selected from the group consisting of cocoyl hydrolyzed silk, potassium cocoyl hydrolyzed silk, sodium cocoyl hydrolyzed silk, isostearoyl hydrolyzed silk, AMP-isostearoyl hydrolyzed silk, sodium lauroyl hydrolyzed silk, sodium stearoyl hydrolyzed silk and mixtures thereof.

6. The process as claimed in claim 1, characterized in that the protein hydrolyzate derivatized with at least one fatty acid is used in the form of a composition which comprises the derivatized protein hydrolyzate in an amount of from 0.05 to 20% by weight based on the total composition.

7. The process as claimed in claim 1, characterized in that the protein hydrolyzate derivatized with at least one fatty acid is used in the form of an aqueous composition comprising the protein hydrolyzate derivatized with at least one fatty acid and at least one keratin-reducing compound.

8. The process as claimed in claim 7, characterized in that the keratin-reducing compound is selected from thioglycolic acid, thiolactic acid, thiomalic acid, phenylthioglycolic acid, mercaptoethanesulfonic acid and salts and esters thereof, cysteamine, cystein, Bunte salts and salts of sulfurous acid, alkali metal disulfites, sodium disulfite (Na₂S₂O₅), potassium disulfite (K₂S₂O₅), magnesium disulfite, ammonium disulfite ((NH₄)₂S₂O₅), hydrogen sulfites as alkali metal, magnesium, ammonium or alkanolammonium salts based on a C₂-C₄-mono-, di- or trialkanolamine, and sulfites as alkali metal, ammonium or alkanolammonium salts based on a C₂-C₄-mono-, di- or trialkanolamine.

9. The process as claimed in claim 8, characterized in that the keratin-reducing compound is selected from the group consisting of thioglycolic acid, thiolactic acid and cystein, and salts thereof.

10. The process as claimed in claim 7, characterized in that the keratin-reducing compounds are present in an amount of from 1 to 25% by weight, based on the total composition.

11. A composition for permanently shaping colored keratin fibers, comprising
at least one silk protein hydrolyzate derivatized with at least one fatty acid and
at least one keratin-reducing compound.

12. The composition as claimed in claim 11, characterized in that the protein hydrolyzates are derivatized with at least one C₆-C₃₀-fatty acid.

13. The composition as claimed in claim 11, characterized in that the derivatized protein hydrolyzate is based on a protein of animal origin.

14. The composition as claimed in claim 11, characterized in that the derivatized protein hydrolyzate is a silk protein hydrolyzate.

15. The composition as claimed in claim 11, characterized in that the protein hydrolyzate derivatized with at least one fatty acid is selected from the group consisting of cocoyl hydrolyzed silk, potassium cocoyl hydrolyzed silk, sodium cocoyl hydrolyzed silk, isostearoyl hydrolyzed silk, AMP-isostearoyl hydrolyzed silk, sodium lauroyl hydrolyzed silk, sodium stearoyl hydrolyzed silk and mixtures thereof.

16. The composition as claimed in claim 11, characterized in that the protein hydrolyzate derivatized with at least one fatty acid is used in the form of a composition which comprises the derivatized protein hydrolyzate in an amount of from 0.05 to 20% by weight based on the total composition.

17. The composition as claimed in claim 11, characterized in that the protein hydrolyzate derivatized with at least one fatty acid is used in the form of an aqueous composition comprising the protein hydrolyzate derivatized with at least one fatty acid and at least one keratin-reducing compound.

18. A process for permanently shaping colored fibers containing keratin comprising the step of applying to the fibers a composition comprising
at least one silk protein hydrolyzate derivatized with at least one fatty acid and
at least one keratin-reducing compound.

19. A method of permanently shaping colored fibers containing keratin wherein the fiber, before and/or after mechanical shaping with the help of shaping auxiliaries, is treated with an aqueous, keratin-reducing composition as claimed in claim 11, optionally after a contact time T1 is rinsed with water and/or an aqueous composition, and finally neutralized with an oxidizing composition comprising at least one oxidizing compound, and optionally after a contact time T2 is rinsed and optionally after treated.

20. A method of permanent shaping colored fibers containing keratin wherein
(i) an aqueous, keratin-reducing composition according to claim 11 is applied to the fibers,
(ii) after a contact time T1, the fibers are rinsed and optionally dried,
(iii) the fibers are shaped with the help of shaping auxiliaries, and
(iv) finally an oxidizing composition comprising at least one oxidizing compound, is applied to the fibers and is rinsed off again after a contact time T2.
